(19) United States
(12) Patent Application Publication
(10) Pub. No.: US 2017/0251928 A1
(43) Pub. Date: Sep. 7, 2017

(54) MEASUREMENT DEVICE AND MEASUREMENT METHOD

(71) Applicant: SEIKO EPSON CORPORATION, Tokyo (JP)

(72) Inventor: Taki HASHIMOTO, Shiojiri-shi (JP)

(73) Assignee: SEIKO EPSON CORPORATION, Tokyo (JP)

(21) Appl. No.: 15/438,175

(22) Filed: Feb. 21, 2017

(30) Foreign Application Priority Data
Mar. 4, 2016 (JP) 2016-042380

(51) Int. Cl. A61B 5/0205; A61B 5/1455; A61B 5/00

(52) U.S. Cl. CPC A61B 5/0205; A61B 5/4809; A61B 5/7278; A61B 5/742; A61B 5/681; A61B 5/14552; A61B 5/74

(57) ABSTRACT

A measurement device includes a measurement unit that sequentially identifies a measurement value of oxygen saturation from a pulse wave signal indicating a pulse wave of an examinee, a setting unit that sets a reference range of the oxygen saturation of the examinee in accordance with the measurement value identified by the measurement unit, an analysis unit that compares the measurement value identified by the measurement unit and the reference range set by the setting unit with each other after the reference range is set, and a notification unit that notifies a measurement result corresponding to a comparison result obtained by the analysis unit.

### BACKGROUND

1. Technical Field

The present invention relates to a technique for measuring oxygen saturation (SpO2) in arterial blood.

2. Related Art

For example, oxygen saturation in arterial blood is an important index used in order to detect disorders (for example, diseases such as pneumonia and asthma) in a respiratory function. In the related art, various techniques are known which relate to noninvasive measurement of the oxygen saturation and notification of the measurement result. For example, JP-A-2005-279262 discloses a technique in which a temporal change in an examinee's lug volume and a temporal change in the oxygen saturation are displayed in parallel. In addition, JP-A-2015-136568 discloses a technique for discriminating a state of the cardiopulmonary and the blood vessel in accordance with the concentration of carbon dioxide and the oxygen saturation in a breath.

Incidentally, an individual difference is present in the oxygen saturation. For example, even in a normal state where an aged person has no particular disorder in his or her respiratory function, functional deterioration occurs due to aging. As a result, in some cases, a numerical value of the oxygen saturation becomes lower. Therefore, even if whether the oxygen saturation is high or low is discriminated based on a predetermined fixed value, there is a problem in that the respiratory function cannot always be properly evaluated.

### SUMMARY

An advantage of some aspects of the invention is to properly evaluate oxygen saturation of an examinee.

A measurement device according to a preferred aspect of the invention includes a measurement unit that sequentially identifies a measurement value of oxygen saturation from a pulse wave signal indicating a pulse wave of an examinee, a setting unit that sets a reference range of the oxygen saturation of the examinee in accordance with the measurement value identified by the measurement unit, an analysis unit that compares the measurement value identified by the measurement unit and the reference range set by the setting unit with each other after the reference range is set, and a notification unit that notifies a measurement result corresponding to a comparison result obtained by the analysis unit. In the above-described aspect, the reference range of the oxygen saturation of the examinee is set in accordance with the measurement value identified by the measurement unit, and the measurement result corresponding to the comparison result between the measurement value identified by the measurement unit and the reference range is notified. Therefore, regardless of every examinee's difference in the oxygen saturation, the oxygen saturation of the examinee can be properly evaluated.

In the preferred aspect of the invention, as the measurement result, the notification unit may notify abnormality in the oxygen saturation, in a case where the measurement value is a numerical value beyond the reference range for a predetermined period of time. In the above-described aspect, the abnormality of the oxygen saturation is notified in a case where the measurement value is the numerical value beyond the reference range for the predetermined period of time. Therefore, a possibility of an unexpected measurement error is minimized, thereby achieving an advantageous effect in that the oxygen saturation of the examinee can be properly evaluated.

In the preferred aspect of the invention, the notification unit may notify the other device of the measurement result. In the above-described aspect, the measurement result of the oxygen saturation of the examinee is notified to a device separate from the measurement device. Therefore, even in a case where the examinee cannot recognize the measurement result, an advantageous effect is achieved in that a user of the other device (for example, a family member of the examinee or a related person such as a caregiver) can understand the measurement result.

In the preferred aspect of the invention, when the setting unit sets the reference range, the notification unit may notify the measurement result, in a case where the measurement value identified by the measurement unit is a numerical value beyond a predetermined range. In the above-described aspect, when the reference range is set, the measurement value is notified in a case where the measurement value identified by the measurement unit is the numerical value beyond the predetermined range. Therefore, the reference range can be properly set by restraining a measurement error from occurring unexpectedly.

In the preferred aspect of the invention, the setting unit may calculate a dispersion degree of multiple measurement values identified by the measurement unit, and may set the reference range, based on the measurement value within an observation period of a variable length corresponding to the dispersion degree. In the above-described aspect, the observation period is variably set in accordance with the dispersion degree of the multiple measurement values (that is, a stable index of the multiple measurement values). Therefore, an advantageous effect is achieved in that the measurement value needed to properly set the reference range is restrained from being excessively sufficient or insufficient.

The measurement device in the preferred aspect of the invention may further include a sleeping determination unit that determines whether or not the examinee is in a sleeping state. The setting unit sets the reference range, based on the measurement value within an observation period during which the sleeping determination unit determines that the examinee is in the sleeping state. In the above-described aspect, the reference range is set based on the measurement value within the observation period during which it is determined that the examinee is in the sleeping state. Accordingly, with regard to the measurement value within the observation period, a measurement error is restrained from occurring due to a physical exercise of the examinee. Therefore, it is possible to set a proper reference range which reflects a steady tendency of the oxygen saturation of the examinee.

The measurement device in the preferred aspect of the invention may further include a detection unit that generates a pulse wave signal indicating a pulse wave of the examinee. The measurement unit may sequentially identify the oxygen saturation from the pulse wave signal generated by the detection unit. The measurement device can be installed on a wrist of the examinee. In the above-described aspect, the measurement device can be installed on the wrist of the examinee. Therefore, the oxygen saturation can be measured at all times without hindering a daily life of the examinee.

A measurement method according to another preferred aspect of the invention causes a computer to sequentially identify a measurement value of oxygen saturation from a pulse wave signal indicating a pulse wave of an examinee, to set a reference range of the oxygen saturation in accordance with the measurement value, to compare the measurement value identified after the reference range is set and the reference range with each other, and to notify a measurement result in accordance with the comparison result. In the above-described method, the reference range of the oxygen saturation of the examinee is set in accordance with the measurement value identified from the pulse wave signal, and the measurement result corresponding to the comparison result between the measurement value identified after the reference range is set and the reference range is notified. Therefore, regardless of every examinee's difference in the oxygen saturation, the oxygen saturation of the examinee can be properly evaluated.

### BRIEF DESCRIPTION OF THE DRAWINGS

The invention will be described with reference to the accompanying drawings, wherein like numbers reference like elements.

FIG. 1 is a side view of a measurement device according to a first embodiment of the invention.

FIG. 2 is a functional configuration diagram of the measurement device.

FIG. 3 is a flowchart of a reference setting process.

FIG. 4 is a view for describing the reference setting process.

FIG. 5 is a schematic view of a measurement result screen.

FIG. 6 is a schematic view of the measurement result screen.

FIG. 7 is a schematic view of the measurement result screen.

FIG. 8 is a schematic view of the measurement result screen.

FIG. 9 is a flowchart of a measurement process.

FIG. 10 is a configuration diagram of a measurement device according to a second embodiment.

FIG. 11 is a flowchart of a reference setting process according to a third embodiment.

FIG. 12 is a flowchart of a reference setting process according to a fourth embodiment.

FIG. 13 is a configuration diagram of a measurement device according to a fifth embodiment.

FIG. 14 is a flowchart of a reference setting process according to the fifth embodiment.

FIG. 15 is a configuration diagram of a measurement device according to a sixth embodiment.

FIG. 16 is a configuration diagram of a measurement device according to a modification example of the sixth embodiment.

### DESCRIPTION OF EXEMPLARY EMBODIMENTS

### First Embodiment

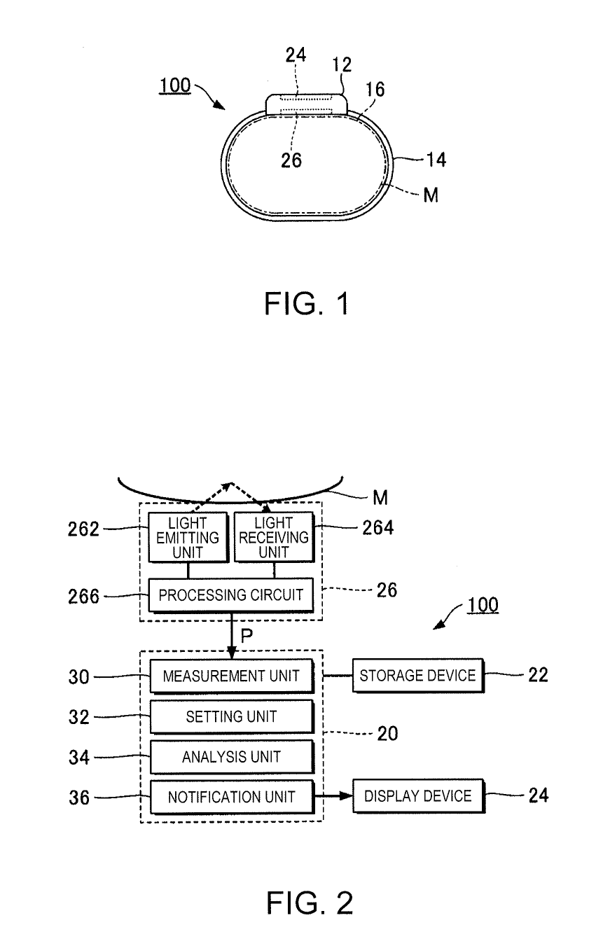

FIG. 1 is a side view of a measurement device 100 according to a first embodiment of the invention. The measurement device 100 according to the first embodiment is a biometric device (oxygen saturation measurement device) for noninvasively measuring oxygen saturation (SpO2) of an examinee, and is installed on a measurement target site (hereinafter, referred to as a “measurement site”) M in a body of the examinee. The measurement device 100 according to the first embodiment is a wristwatch-type portable device including a housing unit 12 and a belt 14, and can be installed on a wrist of the examinee by winding the belt 14 around the wrist serving as an example of the measurement site M. The measurement device 100 according to the first embodiment comes into contact with a surface 16 of the wrist of the examinee.

FIG. 2 is a configuration diagram focusing on a function of the measurement device 100 according to the first embodiment. As illustrated in FIG. 2, the measurement device 100 according to the first embodiment includes a control device 20, a storage device 22, a display device 24, and a detection device 26. The control device 20 and the storage device 22 are installed inside the housing unit 12. As illustrated in FIG. 1, the display device 24 (for example, a liquid crystal display panel) is installed on a surface (for example, a surface opposite to the measurement site M) of the housing unit 12, and displays various images including measurement results under the control of the control device 20.

The detection device 26 (example of a detection unit) in FIG. 2 is an optical sensor which generates a pulse wave signal P indicating a pulse wave of the examinee. For example, the detection device 26 is installed on a facing surface (hereinafter, referred to as a “detection surface”) of the housing unit 12 which faces the measurement site M. The detection device 26 according to the first embodiment includes a light emitting unit 262, a light receiving unit 264, and a processing circuit 266.

For example, the light emitting unit 262 is configured to include a light emitting element such as a light emitting diode (LED), and emits light to the measurement site M from the detection surface of the housing unit 12. The light emitting unit 262 according to the first embodiment includes multiple light emitting elements which can emit the light having different wavelengths. For example, the light emitting unit 262 can emit red light and near-infrared light. For example, the light receiving unit 264 is configured to include a light receiving element such as a photodiode, and generates an output signal corresponding to a received light amount by receiving the light which reaches the detection surface after the light is emitted by the light emitting unit 262 and passes through the inside of the measurement site M. In FIG. 2, a reflection-type optical sensor in which both the light emitting unit 262 and the light receiving unit 264 are installed on the detection surface is illustrated as an example of the detection device 26. However, as the detection device 26, it is also possible to employ a transmission-type optical sensor in which the light emitting unit 262 and the light receiving unit 264 are located on sides opposite to each other across the measurement site M.

The processing circuit 266 drives the light emitting unit 262 and the light receiving unit 264 so as to generate the pulse wave signal P. Specifically, the processing circuit 266 causes the light emitting unit 262 to emit light, and generates the digital pulse wave signal P in such a way that the light receiving unit 264 performs A/D conversion on an output signal generated corresponding to a received light amount when the light emitting unit 262 emits the light. For example, the processing circuit 266 is configured to include a drive circuit which drives the light emitting unit 262 by supplying a drive current, and an output circuit which amplifies and performs A/D conversion on the output signal of the light receiving unit 264 (for example, an amplifier circuit and an A/D converter).

The control device 20 in FIG. 2 is an arithmetic processing unit such as a central processing unit (CPU) and a field-programmable gate array (FPGA), and controls the overall measurement device 100. For example, the storage device 22 is configured to include a nonvolatile semiconductor memory, and stores programs to be executed by the control device 20 or various data items to be used by the control device 20. The control device 20 according to the first embodiment executes the program stored in the storage device 22, thereby realizing multiple functions (a measurement unit 30, a setting unit 32, an analysis unit 34, and a notification unit 36) for measuring and evaluating the oxygen saturation of the examinee. It is possible to employ a configuration in which the functions of the control device 20 is distributed to multiple integrated circuits or a configuration in which the functions of the control device 20 is partially or entirely realized by a dedicated electronic circuit. In FIG. 2, the control device 20 and the storage device 22 are illustrated as separate elements. However, for example, the control device 20 including the storage device 22 can also be realized by an application specific integrated circuit (ASIC).

The measurement unit 30 sequentially identifies a numerical value (hereinafter, referred to as a “measurement value”) X of the oxygen saturation of the examinee from the pulse wave signal P generated by the detection device 26. The measurement value X of the oxygen saturation means a proportion (%) of hemoglobin bound to oxygen in hemoglobin contained in the blood of the examinee, and is an index for evaluating a respiratory function of the examinee. In order to identify the measurement value X, it is possible to optionally employ a known technique. For example, it is possible to identify measurement value X from a ratio Φ (Φ=C2/C1) of a component ratio C2 to a component ratio C1. For example, the component ratio C1 is an intensity ratio between a variation component and a steady component of the pulse wave signal P when the light emitting unit 262 emits near-infrared light. For example, the component ratio C2 is an intensity ratio between the variation component and the steady component of the pulse wave signal P when the light emitting unit 262 emits red light. For example, the measurement unit 30 refers to a table in which each numerical value of the proportion Φ and each numerical value of the measurement value X are associated with each other, and sequentially identifies the measurement value X of the oxygen saturation from the pulse wave signal P. It is also possible to calculate the measurement value X by performing predetermined calculation to which the proportion Φ is applied.

The setting unit 32 sets a reference range R of the oxygen saturation of the examinee. The reference range R means a range of the oxygen saturation when the examinee is in a healthy state (that is, a normal range), and may vary depending on each examinee. The setting unit 32 according to the first embodiment sets the reference range R of the examinee in accordance with the measurement value X sequentially identified by the measurement unit 30.

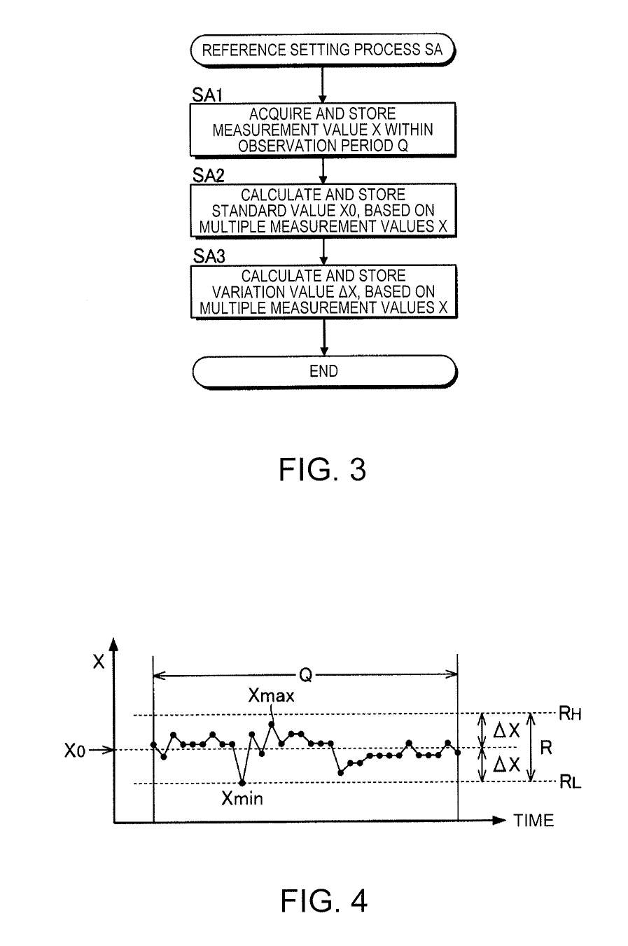

FIG. 3 is a flowchart of a process SA in which the setting unit 32 sets the reference range R of the examinee (hereinafter, referred to as a “reference setting process”), and FIG. 4 is a view for describing the reference setting process SA. For example, immediately after a doctor diagnoses that the examinee is in a healthy state, the reference setting process SA in FIG. 3 is performed in accordance with an instruction from the examinee, as a trigger.

If the reference setting process SA starts, as illustrated in FIG. 4, the setting unit 32 acquires the measurement value X sequentially identified by the measurement unit 30 for a predetermined period of time (hereinafter, referred to as an “observation period”) Q, and stores the measurement value X in the storage device 22 (SA1). For example, the observation period Q means a period ranging from several minutes to several hours. Within the observation period Q, the examinee maintains a tranquil state. It is also possible to discard an abnormal value (for example, a numerical value below 70% or a numerical value above 100%) of the measurement value X.

If the observation period Q elapses, the setting unit calculates a representative value of the multiple measurement values X within the observation period Q as a standard value X0, and stores the representative value in the storage device 22 (SA2). Specifically, an average value or a median value of the multiple measurement values X within the observation period Q is calculated as the standard value X0. The setting unit 32 calculates a variation value ΔX, based on the multiple measurement values X within the observation period Q, and stores the variation value ΔX in the storage device 22 (SA3). As illustrated in FIG. 4, for example, the variation value ΔX is a difference between a minimum value Xmin of the multiple measurement values X within the observation period Q and the standard value X0 (ΔX=X0−Xmin).

For example, it is also possible to employ a configuration in which a numerical value corresponding to a standard deviation (SD) of the multiple measurement values X within the observation period Q (for example, 1SD or 2SD) is calculated as the variation value ΔX, or a configuration in which a fixed value set in advance or a variable value in accordance with a user's instruction is set as the variation value ΔX. The numerical value obtained by subtracting the variation value ΔX from the standard value X0 corresponds to a lower limit value RL (RL=X0−ΔX) of the reference range R, and the numerical value obtained by adding the variation value ΔX to the standard value X0 corresponds to an upper limit value RH (RH=X0+ΔX) of the reference range R. Based on the multiple measurement values X within the observation period Q, the upper limit value RH (for example, a maximum value X max of the measurement value X) and the lower limit value RL (for example, the minimum value Xmin) within the reference range R can be calculated and stored in the storage device 22.

The analysis unit 34 in FIG. 2 determines whether or not the measurement value X identified by the measurement unit 30 after the reference setting process SA is performed is a numerical value (RL≦X≦RH) within the reference range R set by the setting unit 32 in the reference setting process SA. The measurement value X is sequentially determined by the analysis unit 34 for each of the multiple measurement values X identified by the measurement unit 30 after the reference range R is set by performing the reference setting process SA.

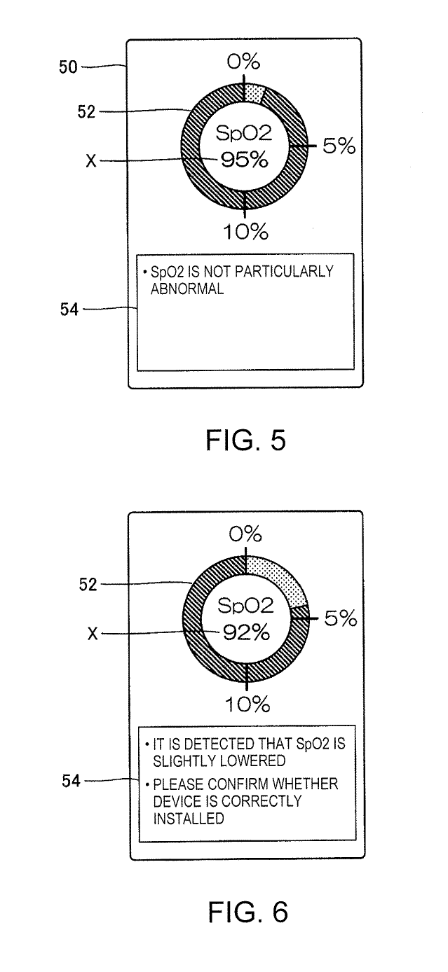

The notification unit 36 notifies a user (the examinee or a related person thereof) of a measurement result of the oxygen saturation of the examinee. The notification unit 36 according to the first embodiment causes the display device 24 to display a screen 50 in FIG. 5 which displays the measurement result (hereinafter, referred to as a “measurement result screen”). For example, the measurement unit 30 generates image data displaying the measurement result screen 50, and supplies the image data to the display device 24, thereby displaying the measurement result screen 50.

As illustrated in FIG. 5, the measurement result screen 50 includes the measurement value X identified by the measurement unit 30, a variation image 52, and an evaluation comment 54. The variation image 52 is an image representing variations in the measurement value X with respect to the standard value X0. The variation image 52 according to the first embodiment has an annular figure (circular graph) which surrounds the measurement value X, and is divided into two regions along the circumferential direction. A ratio of the circumference between the two regions is set in accordance with an absolute value |X0−X| of the difference between the standard value X0 and the measurement value X. The variation image 52 in which the variations in the measurement value X with respect to the standard value X0 are expressed using a figure is illustrated in FIG. 5. However, the variations in the measurement value X can also be displayed using a numerical value (for example, a difference value between the standard value X0 and the measurement value X).

The evaluation comment 54 is a character string (for example, advice, recommendation, proposal, or warning) expressing the measurement result of the oxygen saturation (measurement value X) of the examinee. In accordance with a result obtained by the analysis unit 34 comparing the measurement value X and the reference range R with each other, content of the evaluation comment 54 varies. Specifically, in a case where the analysis unit 34 determines that the measurement value X is the numerical value within the reference range R (RL≦X≦RH), as illustrated in FIG. 5, the evaluation comment 54 meaning that there is no abnormality in the oxygen saturation of the examinee (for example, a character string of “SpO2 is not particularly abnormal”) is displayed on the display device 24.

FIGS. 6 to 8 illustrate each display example of the measurement result screen 50 in a case where the analysis unit 34 determines that the measurement value X is a numerical value beyond the reference range R (X<RL and RH<X). In a case where the measurement value X varies to be beyond the reference range R, as illustrated in FIG. 6, the notification unit 36 causes the display device 24 to display the evaluation comment 54 meaning the variations of the oxygen saturation (for example, a character string of “It is detected that SpO2 is slightly lowered”). If the measurement value X varies to be a numerical value beyond the reference range R, there is a possibility of an unexpected measurement error caused by the physical exercise (body movement) of the examinee or position misalignment of the detection device 26, for example. Therefore, at a stage immediately after the measurement value X varies to be the numerical value beyond the reference range R, as illustrated in FIG. 6, the notification unit 36 according to the first embodiment causes the display device 24 to display the evaluation comment 54 meaning a possibility that the oxygen saturation cannot be properly measured (for example, a character string of “Please confirm whether the device is correctly installed”).

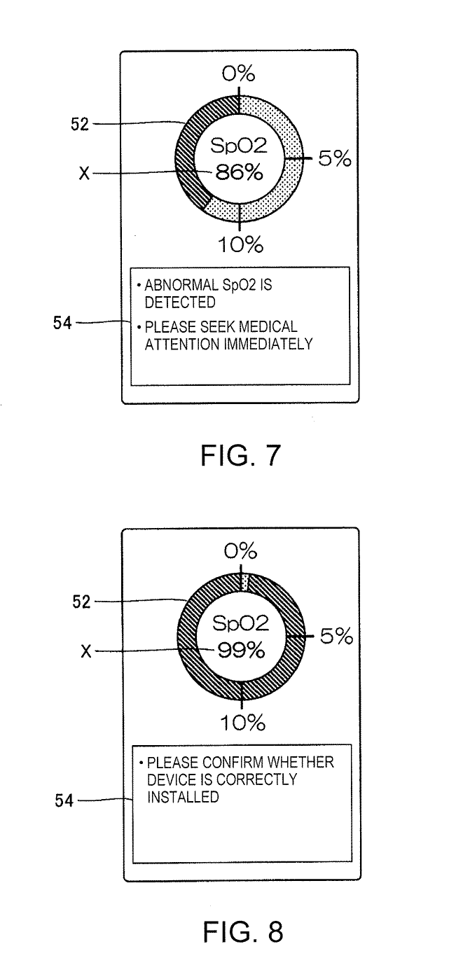

On the other hand, if a state where the measurement value X is the numerical value beyond the reference range R is continuous for a predetermined period of time, it can be estimated that the measurement value X varies due to the abnormal respiratory function of the examinee. Therefore, in a case where the measurement value X is continuously below the lower limit value RL in the reference range R (X<RL) for the predetermined period of time, as illustrated in FIG. 7, the notification unit 36 causes the display device 24 to display the evaluation comment 54 notifying the abnormal oxygen saturation of the examinee as the measurement result. In FIG. 7, a case is illustrated where the evaluation comment 54 of “Abnormal SpO2 is detected. Please seek medical attention immediately”. As can be understood from the above description, according to the first embodiment, in a case where the measurement value X is the numerical value beyond the reference range R, the content of the evaluation comment 54 is changed before the predetermined period of time elapses (FIG. 6) and after the predetermined period of time elapses (FIG. 7).

In a case where the measurement value X is above the upper limit value RH in the reference range R (X>RH), it is possible to evaluate that there is no abnormality in the respiratory function of the examinee. However, there is a possibility of a measurement error caused by the position misalignment of the detection device 26. Therefore, in a case where the measurement value X is continuously above the upper limit value RH in the reference range R for the predetermined period of time, as illustrated in FIG. 8, the notification unit 36 causes the display device 24 to display the evaluation comment 54 meaning a possibility that the oxygen saturation cannot be properly measured due to an installation error of the measurement device 100 (for example, a character string of “Please confirm whether the device is correctly installed”).

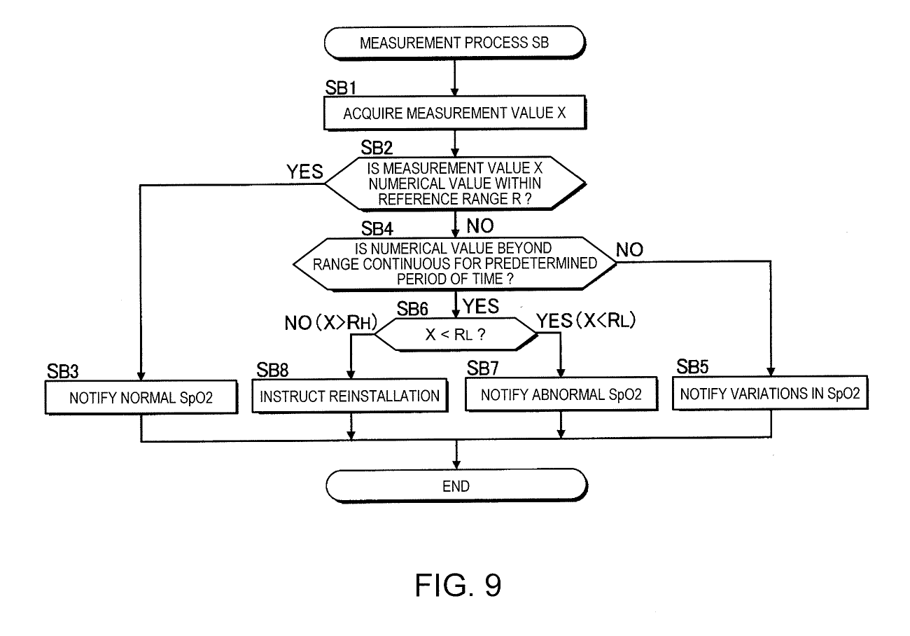

FIG. 9 is a flowchart of a process SB for measuring and notifying the oxygen saturation of the examinee (hereinafter, referred to as a “measurement process”). After the reference setting process SA illustrated in FIG. 3 is performed (after reference range R of the examinee is set), the measurement process SB in FIG. 9 is periodically performed each time that the measurement unit 30 identifies the measurement value X.

If the measurement process SB starts, the analysis unit 34 acquires the measurement value X identified by the measurement unit 30 (SB1), and compares the reference range R set in the reference setting process SA and the measurement value X with each other (SB2). Specifically, the analysis unit 34 determines whether or not the measurement value X is the numerical value within the reference range R. In a case where the measurement value X is the numerical value within the reference range R (SB2: YES), the notification unit 36 causes the display device 24 to display the measurement result screen 50 (FIG. 5) including the measurement value X, the variation image 52, and the evaluation comment 54 notifying that there is no abnormality in the oxygen saturation (SB3).

On the other hand, in a case where the measurement value X is the numerical value beyond the reference range R (SB2: NO), the notification unit 36 determines whether or not a state where the measurement value X is the numerical value beyond the reference range R is continuous for the predetermined period of time (SB4). In a case where the state where the measurement value X is the numerical value beyond the reference range R is not continuous for the predetermined period of time (SB4: NO), the notification unit 36 causes the display device 24 to display the measurement result screen 50 (FIG. 6) including the measurement value X, the variation image 52, and the evaluation comment 54 notifying the variations in the oxygen saturation and a possibility of the measurement error (SB5). On the other hand, in a case where the state where the measurement value X is the numerical value beyond the reference range R is continuous for the predetermined period of time (SB4: YES), the notification unit 36 determines whether or not the measurement value X is below the lower limit value RL in the reference range R (SB6). In a case where the measurement value X is below the lower limit value RL in the reference range R (SB6: YES), the notification unit 36 causes the display device 24 to display the measurement result screen 50 (FIG. 7) including the measurement value X, the variation image 52, and the evaluation comment 54 notifying the abnormal oxygen saturation (SB7). On the other hand, in a case where the measurement value X is above the lower limit value RL in the reference range R (SB6: NO), that is, in a case where the measurement value X is above the upper limit value RH in the reference range R (X>RH), the notification unit 36 causes the display device 24 to display the measurement result screen 50 (FIG. 8) including the measurement value X, the variation image 52, and the evaluation comment 54 instructing reinstallation in order to rectify the installation error (SB8).

As described above, according to the first embodiment, in accordance with the measurement value X measured by the measurement unit 30, the reference range R of the oxygen saturation of the examinee is variably set so as to notify the measurement result corresponding to the comparison result between the measurement value X identified by the measurement unit 30 and the reference range R. Therefore, regardless of every examinee's difference in the oxygen saturation, the oxygen saturation of the examinee can be properly evaluated. As described above, the respiratory function of the examinee can be properly evaluated. As a result, according to the first embodiment, the abnormal respiratory function (for example, diseases such as pneumonia and asthma) of the examinee can be detected at an early stage. In this manner, it is possible to restrain the diseases from becoming severer.

According to the first embodiment, the abnormal oxygen saturation is notified in a case where the measurement value X is continuously the numerical value beyond the reference range R for the predetermined period of time. Therefore, a possibility of an unexpected measurement error caused by the physical exercise (body movement) of the examinee or position misalignment of the detection device 26 is minimized, thereby achieving an advantageous effect in that the oxygen saturation of the examinee can be properly evaluated.

### Second Embodiment

A second embodiment of the invention will be described. In each embodiment described below as an example, the reference numerals used in describing the first embodiment will be given to elements having an operation effect and a function which are the same as those according to the first embodiment, and detailed description of each element will be appropriately omitted.

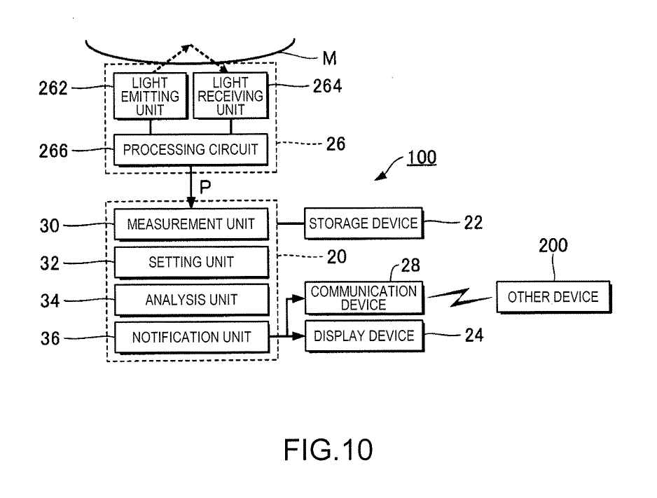

FIG. 10 is a configuration diagram of the measurement device 100 according to the second embodiment. As illustrated in FIG. 10, the measurement device 100 according to the second embodiment includes a communication device 28 in addition to elements the same as those according to the first embodiment. The communication device 28 is a wireless communication device which wirelessly communicates with a different device (hereinafter, referred to as the “other device”) 200 which is separate from the measurement device 100. For example, the communication device 28 communicates with the other device 200 by using short-range wireless communication such as Bluetooth (registered trademark), Wi-Fi (registered trademark), and infrared communication. However, a communication method using the communication device 28 is optionally employed. For example, the communication device 28 can communicate with the other device 200 via a communication network such as a mobile communication network and the Internet. The other device 200 is a terminal device such as a mobile phone and a smartphone owned by a related person of the examinee (for example, a family member or a caregiver), and includes a display device (for example, a liquid crystal display panel) for displaying an image.

Similarly to the first embodiment, the measurement value X is identified by the measurement unit 30, the reference range R is set by the setting unit 32, and the measurement value X is compared with the reference range R by the analysis unit 34. The notification unit 36 according to the second embodiment notifies the other device 200 of the measurement result corresponding to the result obtained by the analysis unit 34 comparing the measurement value X of the examinee and the reference range R with each other. Specifically, similarly to the first embodiment, the notification unit 36 causes the display device 24 to display the measurement result screen 50. In addition, the notification unit 36 transmits image data displaying the measurement result screen 50 from the communication device 28 to the other device 200, thereby causing the display device of the other device 200 to display the measurement result screen 50. Display of the measurement result screen 50 using the display device 24 of the measurement device 100 can be omitted.

According to the second embodiment, it is also possible to realize an advantageous effect which is the same as that according to the first embodiment. Incidentally, for example, in a case where there is a disorder in a cognitive function of the examinee, even if the measurement result is displayed on the display device 24 of the measurement device 100 installed for the examinee, a possibility that the examinee himself or herself may not notice the notification of the abnormal oxygen saturation is assumed. For example, in a case where the examinee is an infant, the examinee himself or herself cannot recognize that the abnormal oxygen saturation is notified by the display device 24. According to the second embodiment, the measurement result of the oxygen saturation of the examinee is notified to the other device 200 separate from the measurement device 100 installed for the examinee. Therefore, even in a case where the examinee cannot recognize the measurement result, there is an advantageous effect in that a related person holding the other device 200 can understand the measurement result (for example, the abnormal oxygen saturation).

It is preferable to adopt a configuration in which the communication device 28 transmits the measurement result to the other device 200 registered in advance as a transmission destination. However, without designating the transmission destination, the measurement result can also be transmitted to the other device 200 around the measurement device 100.

### Third Embodiment

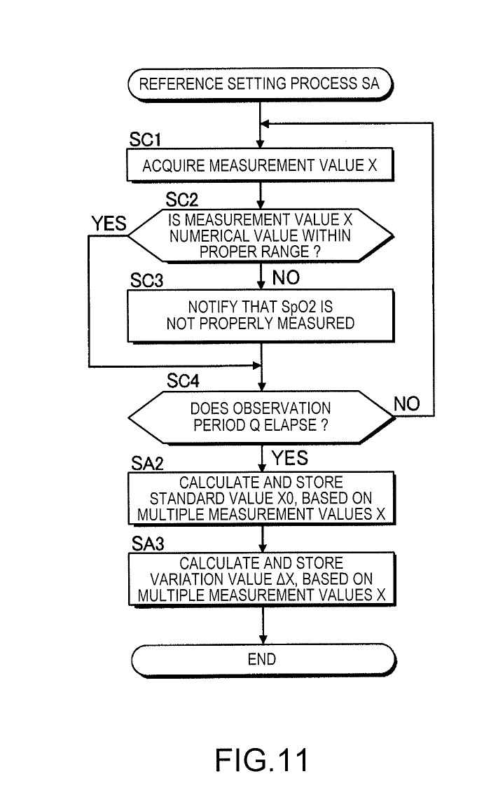

FIG. 11 is a flowchart of the reference setting process SA according to a third embodiment. As illustrated in FIG. 11, in the reference setting process SA according to the third embodiment, Step SA1 in the reference setting process SA according to the first embodiment illustrated in FIG. 3 is replaced with Steps SC1 to SC4 in FIG. 11.

As illustrated in FIG. 11, if the reference setting process SA starts, the setting unit 32 according to the third embodiment acquires the measurement value X identified by the measurement unit 30 (SC1), and determines whether or not the measurement value X is a numerical value within a predetermined range (hereinafter, referred to as a “proper range”) (SC2). For example, the proper range is a variable range corresponding to the measurement value X in the past. Specifically, a range of a predetermined width including an average value (for example, average value ±2%) of the multiple measurement values X identified by the measurement unit 30 in the past while the reference setting process SA is performed is set as the proper range. The proper range is set so that the proper measurement value X measured in a state where there is no measurement error caused by the physical exercise of the examinee or position misalignment of the detection device 26 is the numerical value within the proper range (so that the measurement value X measured in a state where the measurement error occurs is the numerical value beyond the proper range).

In a case where the measurement value X is the numerical value beyond the proper range (SC2: NO), the notification unit 36 notifies the examinee of a fact that the oxygen saturation cannot be properly measured (that is, a fact that the measurement value X is an improper numerical value) (SC3). Specifically, the notification unit 36 causes the display device 24 to display a comment meaning that the oxygen saturation cannot be properly measured (for example, a character string of “Please check an installation state of the device and keep your body tranquil”). If through the notification from the notification unit 36, the examinee comes to understand that the oxygen saturation cannot be properly measured, the examinee stops his or her own body movement or adjusts a position of the detection device 26 (installation state of the measurement device 100), thereby adjusting the existing state to a state where the oxygen saturation can be properly measured. As a result of the adjustment by the examinee, the subsequent measurement value X is maintained to be the numerical value within the proper range. On the other hand, in a case where the measurement value X is the numerical value within the proper range (SC2: YES), the notification unit 36 does not perform the notification. In a case where the measurement value X is the numerical value within the proper range, the notification unit 36 can also notify the examinee of the fact that the oxygen saturation can be properly measured.

The above-described process is repeated until the observation period Q elapses (SC4: NO). If the observation period Q elapses (SC4: YES), similarly to the first embodiment, the setting unit 32 sets the reference range R in accordance with the multiple measurement values X within the observation period Q (SA2 and SA3). The measurement value X beyond the proper range out of the multiple measurement values X within the observation period Q can also be excluded from a target of calculation of the standard value X0 (SA2) or calculation of the variation value ΔX (SA3).

According to the third embodiment, it is also possible to realize an advantageous effect which is the same as that according to the first embodiment. According to the third embodiment, a notification is given in a case where the measurement value X is the numerical value beyond the proper range in the reference setting process SA. Accordingly, for example, it is possible to restrain the measurement error from occurring due to the physical exercise of the examinee or misaligned installation position of the measurement device 100. Therefore, the reference range R can be properly set. The configuration according to the second embodiment in which the measurement result is notified to the other device 200 can be similarly applied to the third embodiment. The notification unit 36 can also use the communication device 28 so as to notify the other device 200 of the fact that the oxygen saturation cannot be properly measured (SC3).

### Fourth Embodiment

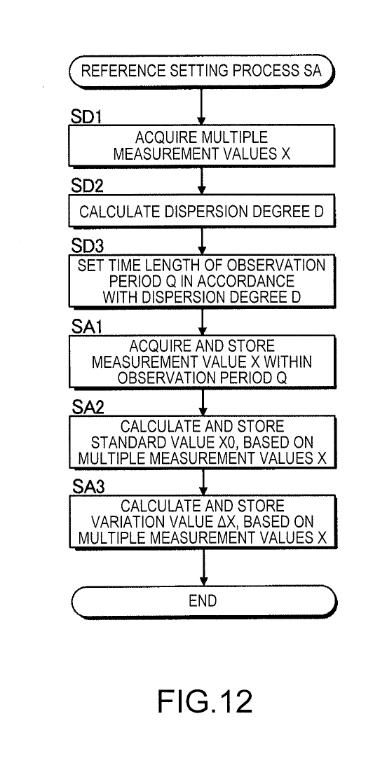

FIG. 12 is a flowchart of the reference setting process SA according to a fourth embodiment. As illustrated in FIG. 12, in the reference setting process SA according to the fourth embodiment, Steps SD1 to SD3 in FIG. 12 are performed prior to Step SA1 in the reference setting process SA according to the first embodiment illustrated in FIG. 3.

As illustrated in FIG. 12, if the reference setting process SA starts, the setting unit 32 according to the fourth embodiment acquires the multiple measurement values X sequentially identified by the measurement unit 30 (SD1). Specifically, the setting unit 32 acquires a predetermined number of the measurement values X identified within a shorter time compared to the observation period Q. The setting unit 32 calculates a dispersion degree D of the multiple measurement values X acquired in Step SD1 (SD2). The dispersion degree D is a statistical index (for example, dispersion or standard deviation) indicating a scattered degree of the multiple measurement values X. As the dispersion degree D is smaller, it can be evaluated that the oxygen saturation of the examinee can be more stably measured.

The setting unit 32 variably sets a time length of the observation period Q in accordance with the dispersion degree D of the multiple measurement values X (SD3). For example, in a case where the dispersion degree D is small, it means that the multiple measurement values X are temporally stable. Therefore, there is a tendency that the reference range R which properly reflects a tendency of the oxygen saturation of the examinee can be set based on the measurement value X within a relatively short time. On the other hand, in a case where the dispersion degree D is large, it means that the multiple measurement values X unstably vary due to the physical exercise of the examinee, for example. Therefore, as long as the measurement value X is not used for a long period of time, there is a tendency that the reference range R cannot be properly set. In view of the above-described tendency, the setting unit 32 according to the fourth embodiment sets the observation period Q to a shorter time as the dispersion degree D is smaller (that is, as the measurement value X is temporally stable).

The setting unit 32 acquires the multiple measurement values X within the observation period Q having the time length set according to the above-described procedure, and stores the multiple measurement values X in the storage device 22 (SA1). Specifically, in Step SA1, the setting unit 32 acquires the multiple measurement values X within the observation period Q which includes the measurement values X previously acquired in Step SD1 described above. Similarly to the first embodiment, the standard value X0 corresponding to the multiple measurement values X within the observation period Q is calculated (SA2), or the variation value ΔX is calculated (SA3).

According to the fourth embodiment, it is also possible to realize an advantageous effect which is the same as that according to the first embodiment. According to the fourth embodiment, the time length of the observation period Q is variably set in accordance with the dispersion degree D of the multiple measurement values X. Therefore, in a situation where the measurement value X is temporally stable (situation where the proper reference range R can be set based on the relatively small number of the measurement values X), it is possible to minimize a possibility that the excessive number of the measurement values X exceeding the limit needed to properly set the reference range R may be used in setting the reference range R. On the other hand, in a situation where the measurement value X temporally and unstably varies (situation where the measurement value X is needed to set the proper reference range R for a long period of time), it is possible to minimize a possibility that the number of the measurement values X used in setting the reference range R may be insufficient. That is, according to the fourth embodiment, an advantageous effect is achieved in that the measurement value X needed to properly set the reference range R is restrained from being excessively sufficient or insufficient.

The configuration according to the second embodiment in which the measurement result is notified to the other device 200 and the configuration according to the third embodiment in which the notification is given in a case where the measurement value X is the numerical value beyond the proper range in the reference setting process SA can be similarly applied to the fourth embodiment.

### Fifth Embodiment

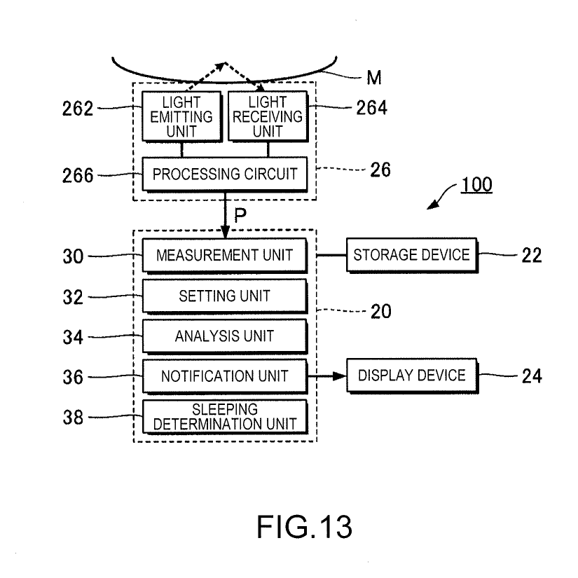

FIG. 13 is a configuration diagram illustrating the measurement device 100 according to a fifth embodiment. As illustrated in FIG. 13, the control device 20 of the measurement device 100 according to the fifth embodiment functions as a sleeping determination unit 38 in addition to elements which are the same as those according to the first embodiment (the measurement unit 30, the setting unit 32, the analysis unit 34, and the notification unit 36). The sleeping determination unit 38 determines whether or not the examinee is in a sleeping state (sleeping state/awakening state).

Specifically, the sleeping determination unit 38 according to the fifth embodiment analyzes the pulse wave signal P generated by the detection device 26 so as to determine the sleeping state/awakening state of the examinee. In order to analyze the sleeping by using the pulse wave signal P, it is possible to optionally employ a known technique. For example, a relationship is analyzed between a low frequency component (for example, 0.04 to 0.15 Hz) reflecting sympathetic and parasympathetic activities in the pulse wave signal P and a high frequency component (for example, 0.15 to 0.4 Hz) reflecting the parasympathetic activity. In this manner, it is possible to determine the sleeping state/awakening state of the examinee.

In the sleeping state where the body of the examinee is tranquil, for example, the measurement error is restrained from occurring due to the physical exercise of the examinee. Accordingly, there is a tendency that the multiple measurement values X of the oxygen saturation are stably identified. Therefore, the sleeping state is more preferably used than the awakening state in setting the proper reference range R reflecting a steady tendency of the oxygen saturation. In view of the above-described circumstances, the setting unit 32 according to the fifth embodiment sets the reference range R, based on the multiple measurement values X identified in a state where the sleeping determination unit 38 determines that the examinee is in the sleeping state.

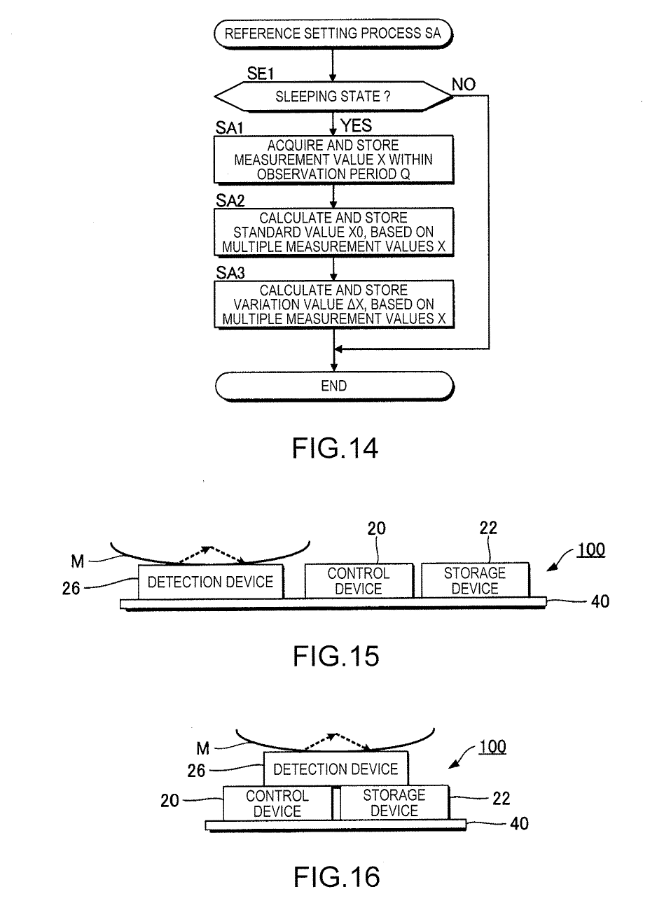

FIG. 14 is a flowchart of the reference setting process SA according to the fifth embodiment. As illustrated in FIG. 14, in the reference setting process SA according to the fifth embodiment, Step SE1 in FIG. 14 is performed prior to Step SA1 in the reference setting process SA according to the first embodiment illustrated in FIG. 3.

As illustrated in FIG. 14, if the reference setting process SA starts, the sleeping determination unit 38 determines whether or not the examinee is in the sleeping state (SE1). In a case where the sleeping determination unit 38 determines that the examinee is not in the sleeping state (case where the examinee is in the awakening state), the setting unit 32 completes the reference setting process SA without setting the reference range R (SA1 to SA3) (SE1: NO). On the other hand, in a case where the sleeping determination unit 38 determines that the examinee is in the sleeping state (SE1: YES), similarly to the first embodiment, the setting unit 32 sets the reference range R, based on the multiple measurement values X within the observation period Q (SA1 to SA3). That is, the multiple measurement values X sequentially identified by the measurement unit 30 within the observation period Q during which the examinee is in the sleeping state are employed in setting the reference range R.

According to the fifth embodiment, it is also possible to realize an advantageous effect which is the same as that according to the first embodiment. According to the fifth embodiment, the reference range R is set in accordance with the measurement value X within the observation period Q during which it is determined that the examinee is in the sleeping state. Accordingly, for example, the measurement error is restrained from occurring in the measurement value X within the observation period Q due to the physical exercise of the examinee. Therefore, it is possible to set the proper reference range R which reflects a steady tendency of the oxygen saturation of the examinee.

The configuration according to the second embodiment in which the measurement result is notified to the other device 200 and the configuration according to the third embodiment in which the notification is given in a case where the measurement value X is the numerical value beyond the proper range in the reference setting process SA can be similarly applied to the fifth embodiment. The configuration according to the fourth embodiment in which the time length of the observation period Q is variably set in accordance with the dispersion degree D of the measurement value X can be similarly applied to the fifth embodiment. For example, in a case where the sleeping determination unit 38 determines that the examinee is in the sleeping state (SE1: YES), Steps SD1 to SD3 according to the fourth embodiment (FIG. 12) are performed.

### Sixth Embodiment

In the above-described embodiments, the portable measurement device 100 including the housing unit 12 and the belt 14 has been described as an example. The measurement device 100 according to a sixth embodiment is a measurement module which does not include the housing unit 12 or the belt 14. Specifically, as illustrated in FIG. 15, the measurement device 100 according to the sixth embodiment is an electronic component having a configuration in which the control device 20, the storage device 22, and the detection device 26 are mounted on a board 40 (for example, a wiring board). As illustrated in FIG. 16, it is also preferable to adopt a configuration in which the control device 20 and the storage device 22 are mounted on the board 40, and in which the detection device 26 is disposed at a position closer to the measurement site M compared to the control device 20 and the storage device 22. For example, the measurement device 100 (measurement module) according to the sixth embodiment is assembled to a housing having the display device 24 installed therein, thereby configuring a portable device. Each configuration or each function of the control device 20, the storage device 22, and the detection device 26 is the same as that according to the first embodiment to the fifth embodiment.

### MODIFICATION EXAMPLE

The above-described respective embodiments can be modified in various ways. Hereinafter, specific modification examples will be described. Two or more modification examples optionally selected from the following examples can be appropriately combined with each other.

(1) In a case where the measurement value X is the numerical value beyond the reference range R in the measurement process SB (SB2: NO), content of the evaluation comment 54 can also be changed stepwise in accordance with the measurement value X. For example, in a case where the measurement value X is above a predetermined threshold value XTH beyond the reference range R (XTH<X<RL), the notification unit 36 notifies the evaluation comment 54 showing a message of “The abnormal oxygen saturation is detected. Please seek medical attention immediately”. In a case where the measurement value X is below the predetermined threshold value XTH (X<XTH), the notification unit 36 notifies the evaluation comment 54 showing a message of “It is a dangerous state. Please consider calling an ambulance”.

(2) In the above-described embodiments, the measurement result screen 50 including the measurement value X, the variation image 52, and the evaluation comment 54 is steadily displayed on the display device 24. However, it is also possible to notify a user (the examinee or a related person) of the abnormal oxygen saturation in a limited case where the measurement value X is changed to the numerical value beyond the reference range R. That is, in a state where the measurement value X is the numerical value within the reference range R, the notification of the measurement result (that is, display of the measurement result screen 50) can be omitted.

(3) In the above-described embodiments, the measurement device 100 which can be installed on the wrist of the examinee has been described as an example. However, a specific form (installation position) of the measurement device 100 is optionally selected. For example, it is possible to employ the measurement device 100 having any optional form, such as a patch type which can be attached to the body of the examinee, an earring type which can be worn on auricles of the examinee, a finger wearable type which can be worn on a fingertip of the examinee (for example, a fingernail wearable type), and a head-mounted type which can be mounted on a head of the examinee. However, in a state where the examinee wears the measurement device 100 such as the finger wearable type, it is assumed that there is a possibility that the examinee may feel troublesome in his or her daily life. Accordingly, from a viewpoint that the oxygen saturation is measured at all times without any trouble in the daily life, it is particularly preferable to employ the measurement device 100 according to the above-described respective embodiments which can be installed on the wrist of the examinee. It is also possible to realize the measurement device 100 having a form to be installed in (for example, externally attached to) various electronic devices such as a wristwatch.

(4) In the fifth embodiment, the pulse wave signal P is analyzed so as to determine the sleeping state/awakening state of the examinee. However, a method in which the sleeping determination unit 38 determines whether or not the examinee is in the sleeping state is not limited to the above-described example. For example, the sleeping determination unit 38 can also determine the sleeping state/awakening state, based on a brain wave of the examinee which is detected by a known brain wave sensor or a myoelectric waveform detected by an electromyograph. In the fifth embodiment, the pulse wave signal P generated by the detection device 26 is used for both the identification of the measurement value X of the oxygen saturation and the determination of the sleeping state/awakening state of the examinee. Therefore, compared to a configuration of utilizing a measuring instrument (brain wave sensor or electromyograph) separate from the measurement of the oxygen saturation, an advantageous effect is achieved in that the configuration of the measurement device 100 is simplified.

(5) In the above-described embodiments, the measurement result is displayed on the display device 24. However, a configuration in which the notification unit 36 notifies a user (the examinee or a related person) of the measurement result is not limited to the above-described example. For example, a sound emitting device such as a speaker and an earphone can also notify the user of the measurement result (for example, the measurement value X or the evaluation comment 54) by using a sound. A printing apparatus may notify the user of the measurement result. It is also possible to employ a configuration in which a vibrator notifies the user of abnormality by using vibrations or a configuration in which a light emitting element notifies the user of abnormality by emitting light, in a case where the abnormal oxygen saturation is detected (case where the measurement value X is the numerical value beyond the reference range R).

(6) As described above, the measurement device 100 described as an example in the above-described respective embodiments can be realized by the cooperation of the control device 20 and a program. The program according to a preferred embodiment of the invention causes a computer to execute functions as the measurement unit 30 that sequentially identifies the measurement value X of the oxygen saturation from the pulse wave signal P indicating the pulse wave of the examinee, the setting unit 32 that sets the reference range R of the oxygen saturation in accordance with the measurement value X identified by the measurement unit 30, the analysis unit 34 that compares the measurement value X identified by the measurement unit 30 after the reference range R is set and the reference range R set by the setting unit 32, and the notification unit 36 that notifies the measurement result corresponding to comparison result obtained by the analysis unit 34. The program described above as an example can be provided in a form stored in a computer-readable recording medium, and can be installed in the computer. For example, the recording medium is a non-transitory recording medium, and an optical recording medium (optical disk) such as a CD-ROM is a preferable example. However, any other known optional recording medium such as a semiconductor recording medium and a magnetic recording medium can be included therein. The program can also be distributed to the computer in a distribution form via a communication network.

The entire disclosure of Japanese Patent Application No. 2016-042380 is hereby incorporated herein by reference.

What is claimed is:

1. A measurement device comprising:
a measurement unit that sequentially identifies a measurement value of oxygen saturation from a pulse wave signal indicating a pulse wave of an examinee;
a setting unit that sets a reference range of the oxygen saturation of the examinee in accordance with the measurement value identified by the measurement unit;
an analysis unit that compares the measurement value identified by the measurement unit and the reference range set by the setting unit with each other after the reference range is set; and
a notification unit that notifies a measurement result corresponding to a comparison result obtained by the analysis unit.

2. The measurement device according to claim 1,
wherein as the measurement result, the notification unit notifies abnormality in the oxygen saturation, in a case where the measurement value is a numerical value beyond the reference range for a predetermined period of time.

3. The measurement device according to claim 1,
wherein the notification unit notifies the other device of the measurement result.

4. The measurement device according to claim 1,
wherein when the setting unit sets the reference range, the notification unit notifies the measurement result, in a case where the measurement value identified by the measurement unit is a numerical value beyond a predetermined range.

5. The measurement device according to claim 1,
wherein the setting unit calculates a dispersion degree of multiple measurement values identified by the measurement unit, and sets the reference range, based on the measurement value within an observation period of a variable length corresponding to the dispersion degree.

6. The measurement device according to claim 1, further comprising:
a sleeping determination unit that determines whether or not the examinee is in a sleeping state,
wherein the setting unit sets the reference range, based on the measurement value within an observation period during which the sleeping determination unit determines that the examinee is in the sleeping state.

7. The measurement device according to claim 1, further comprising:
a detection unit that generates a pulse wave signal indicating a pulse wave of the examinee,
wherein the measurement unit sequentially identifies the oxygen saturation from the pulse wave signal generated by the detection unit, and
wherein the measurement device can be installed on a wrist of the examinee.

8. A measurement method causing a computer:
to sequentially identify a measurement value of oxygen saturation from a pulse wave signal indicating a pulse wave of an examinee;
to set a reference range of the oxygen saturation in accordance with the measurement value;
to compare the measurement value identified after the reference range is set and the reference range with each other; and
to notify a measurement result in accordance with the comparison result.
